(19) United States
(12) Patent Application Publication
(10) Pub. No.: US 2025/0054567 A1
(43) Pub. Date: Feb. 13, 2025

(54) MACHINE-LEARNING EXTRACTION OF BIOMEDICAL INFORMATION AND OPTIMIZED CHARACTERIZATION OF A TUMOR MICRO ENVIRONMENT OF A PATIENT

(71) Applicant: NEC Laboratories Europe GmbH, Heidelberg (DE)

(72) Inventors: Giampaolo Pileggi, Heidelberg (DE); Anja Moesch, Heidelberg (DE); Catherine Chaput, Heidelberg (DE); Carolin Lawrence, Heidelberg (DE)

(21) Appl. No.: 18/509,364

(22) Filed: Nov. 15, 2023

Related U.S. Application Data
(60) Provisional application No. 63/518,121, filed on Aug. 8, 2023.

(51) Int. Cl. G16B 5/00; G16B 25/10; G16B 40/20; G16H 50/50; G16H 50/70

(52) U.S. Cl. CPC G16B 5/00; G16B 25/10; G16B 40/20; G16H 50/50; G16H 50/70

(57) ABSTRACT

A computer-implemented machine-learning method characterizes a tumor micro environment. The method includes: using a trained natural language processing machine learning model (NLP-model), extracting facts from biomedical text indicating relationship information between cell types and found gene names; using a reference database having gene names and aliases, grouping the extracted facts according to associated genes to generate extracted and grouped information; and generating a matrix from the extracted and grouped information with a first axis representing cell types and second axis representing genes. Each value of the matrix is calculated based on an importance of an associated gene taken and an associated weight. The associated weight is based associated publication meta information and/or an associated detection method's robustness and reliability. The method has applications including, but not limited to, use cases in drug development, medical artificial intelligence (AI)/healthcare for optimization of predictions or to support decision making.

### CROSS-REFERENCE TO RELATED APPLICATION

Priority is claimed to U.S. Provisional Patent Application No. 63/518,121, filed on Aug. 8, 2023, the entire disclosure of which is hereby incorporated by reference herein.

### FIELD

The present disclosure relates to a machine-learning method, system, and computer-readable medium for biomedical information extraction and optimized characterization of a tumor microenvironment of a patient.

### BACKGROUND

The quest for effective cancer treatments is of paramount importance due to the significant global impact of this devastating disease. Indeed, cancer remains a leading cause of morbidity worldwide, affecting millions of individuals and their families. Despite advancements in medical science, the intricate and heterogeneous nature of cancer prevents a formidable challenge. In other words, it is not easy to determine which treatment is best for someone because that determination often depends on the patient's individual tumor microenvironment—i.e. the composition of cancer and immune cells within the tumor.

Experience has shown that each tumor of each patient differs over time in many different biological parameters, such as at the genetic level of the cancer cells, the cell type composition, and the immune system status of the cancer tissue. Methods developed to be specifically tailored towards these individual tumor features have been shown to be much more efficient than one-size-fits-all approaches. This is particularly true if molecular markers like tumor-specific mutations are used for therapies. These kinds of markers are most often used for immunotherapies, which bring another aspect of uniqueness, because the interaction between tumor and immune system is extremely complex.

In order to apply the most efficient therapy and consider adjuvants, it is crucial to understand the status of the immune system around and within the tumor. This is referred to as the tumor microenvironment (TME), which includes types of tumor infiltrating immune cells, as well as their status, such as exhaustion, in which case they will not be able to fight the tumor regardless of being present inside. Accurately characterizing a patient's TME allows experts to determine which type of treatment should be applied—i.e., patient stratification—and to refine the individual aspects of the treatment, such as immune-stimulating adjuvants for neoantigen cancer vaccines.

To characterize a patient's TME, usually bulk ribonucleic acid (RNA) sequencing is used, which can also be used to determine the expression levels of gene relevant for the immune system's countless functions. However, assigning these gene signatures to individual cell types works only on a high level, without the information single-cell RNA sequencing can provide, and works only by using well-known and established marker genes, which are found and confirmed by laborious manual literature research. See, e.g., Bagaev et al., “Conserved pan-cancer microenvironment subtypes predict response to immunotherapy,” Cancer Cell, 2021 (the entire contents of which is hereby incorporated by reference herein). Such research is time consuming and not comprehensive, given the number of available papers and is also prone to missing unexpected findings, e.g., due to human bias. Therefore, information on how to classify cell types from gene expression data is effectively hidden in a vast amount of scientific publications.

### SUMMARY

An aspect of the present disclosure provides a computer-implemented machine-learning method that characterizes a tumor micro environment. The method includes: using a trained natural language processing machine learning model (NLP-model), extracting facts from biomedical text, the extracted facts indicating relationship information between cell types and found gene names; using a reference database having gene names and gene aliases, grouping the extracted facts according to associated genes to generate extracted and grouped information; and generating a matrix from the extracted and grouped information with a first axis representing cell types and second axis representing genes. Each value of the matrix is calculated based on an importance of an associated gene taken and an associated weight. The associated weight is based on at least one of associated publication meta information or an associated detection method's robustness and reliability. The method has applications including, but not limited to, use cases in drug development, medical artificial intelligence (AI)/healthcare for optimization of predictions or to support decision making.

### BRIEF DESCRIPTION OF THE DRAWINGS

Embodiments of the present disclosure will be described in even greater detail below based on the exemplary figures. The present disclosure is not limited to the exemplary embodiments. All features described and/or illustrated herein can be used alone or combined in different combinations in embodiments of the present disclosure. The features and advantages of various embodiments of the present disclosure will become apparent by reading the following detailed description with reference to the attached drawings which illustrate the following:

FIG. 1 illustrates a method and system according to the present disclosure;

FIG. 2 illustrates an example of extracted information for each cell type; and

FIG. 3 is a block diagram of an exemplary processing system, which can be configured to perform any and all operations disclosed herein.

### DETAILED DESCRIPTION

Aspects of the present disclosure provide a computer-implemented machine-learning tool that automatically characterizes a patient's TME, including extracting information on how to classify cell types from gene expression data with fine-grain resolution. Embodiments implemented according to the present disclosure thereby provide the technological improvement of faster, more complete, more computationally efficient, and more accurate TME characterization, as compared to a manual process executed by human experts. A computer-implemented tool configured according to aspects of the present disclosure can also scan relevant literature available to it automatically and without bias. Such tool will efficiently converge the information into cell-type-specific gene signatures. This allows for a more fine-grained classification of a patient's TME, and helps to identify more targeted treatments.

Thus, the present disclosure provides, for the first time, an automatic computer-implemented system that computationally-efficiently assimilates information from a vast number of medical publications and extracts relevant (often hidden) biomedical information, which enables optimized characterization of the TME of a patient. As a result, classification of which treatment is most appropriate for a certain patient is improved.

According to a first aspect of the present disclosure, a computer-implemented machine learning method is provided for patient stratification based on facts extracted from publications. The method includes at least one of:
1) Selection of publications according to given diseases and cell types;
2) Extraction of text fragments or publication sections expected to contain information;
3) Preprocessing of publication data: such as string matching of gene names, aliases, gene products and associated terms including distinction between human and non-human by using a reference database. This can also be applied to cell type names;
4) Executing an information extraction algorithm, which for each cell type keeps track of: (1) found gene name, (2) free text relation description; and (3) meta information (e.g., including in which publication the gene was found);
5) Determining a grouping of which found genes refer to the same gene via: (1) gene and gene product (e.g., protein) databases; and (2) (optionally) a language model clustering algorithm;
6) Building a matrix by calculating a value for each combination of gene and cell type, including weighting of gene signatures based on metrics indicating reliability (e.g., citations, journal quality, robustness of methods, and confirmation of results by multiple publications);
7) Given single cell expression data from a patient, associate each cell to a cell type and its status or sub-population type by matching the expression pattern to the matrix; and
8) Given single cell expression data from a patient, including the characterization information from step 7, performing stratification (classification) of patient to disease subgroup, treatment response or adjuvant therapy recommendation.

According to a second aspect of the present disclosure, a computer-implemented machine learning method is provided. The method includes at least one of:

1) Executing an information extraction algorithm based on a natural language processing model that is trained to process biomedical texts and can extract meaningful triples, consisting of gene names, relations (associations) and cell types. This includes pre-processing of the input text fragments by matching strings to aliases for genes and cell types.
2) Building the matrix database with one axis representing cell types and one axis representing genes. Each value is calculated by taking the reported importance of the gene into account, as well as applying a weight calculated by taking for example the number of confirming articles, their publication quality and, most important, the detection method's robustness and reliability into account.
3) Expressing data of cell types from patients obtained for stratifying these patients is used to generate a bundled feedback signal to re-adjust weighting of matrix values for existing disease classifications as well as obtaining a higher resolution of subtypes of diseases given detailed clinical diagnosis of said patient.

According to a third aspect of the present disclosure, a computer-implemented machine-learning method is provided that characterizes a tumor micro environment. The method includes: using a trained natural language processing machine learning model (NLP-model), extracting facts from biomedical text, the extracted facts indicating relationship information between cell types and found gene names; using a reference database having gene names and gene aliases, grouping the extracted facts according to associated genes to generate extracted and grouped information; and generating a matrix from the extracted and grouped information with a first axis representing cell types and second axis representing genes. Each value of the matrix is calculated based on an importance of an associated gene taken and an associated weight. The associated weight is based on at least one of associated publication meta information or an associated detection method's robustness and reliability.

In a first implementation of the method according to the third aspect, the method may further include: characterizing the tumor micro environment based on cell expression data of a patient by matching the cell expression data with the matrix.

In a second implementation of the method according to the third aspect, the method of the first implementation may further include: receiving a biological sample of a tumor of the patient; and using ribonucleic acid (RNA) sequencing on the biological sample, generating the cell expression data, which includes respective active gene information associated with each cell of a plurality of cells detected in the biological sample, which in turn corresponds to expression patterns, each respective expression pattern of the expression patterns being associated with a single cell of the cells detected in the biological sample.

In a third implementation of the method according to the third aspect, in the method of the second aspect, characterizing the tumor micro environment based on the cell expression data of the patient by matching the cell expression data with the matrix includes: for each of the cells of the plurality of cells detected in the biological sample: finding a match in the matrix for the respective expression pattern; and assigning the respective cell to one of the cell types of the extracted facts, generating a list of the cell types assigned to the plurality of cells detected in the biological sample; generating cell type fraction data by determining, for each respective cell type of the cell types in the list, a fraction of the respective cell type from among the cell types; outputting the list of the cell types and the cell type fraction data as the tumor micro environment characterization.

In a fourth implementation of the method according to the third aspect, the method of the first implementation may further include updating the matrix based on enriched marker genes found in the biological sample.

In a fifth implementation of the method according to the third aspect, the method of the first implementation may further include: classifying, using the tumor micro environment characterization, the patient to a disease subgroup, a treatment response, adjuvant therapy recommendation, or disease outcome, treatment specification.

According to a sixth implementation of the method according to the third aspect, in the method of the fifth implementation, the classifying may include comparing the tumor micro environment characterization of the patient to historical tumor microenvironment characterizations.

According to a seventh implementation of the method according to the third aspect, in the method of the fifth implementation, the classifying may include using a trained machine-learning classification model to assign the patient to a particular classification using the tumor micro environment characterization as input.

According to an eighth implementation of the method according to the third aspect, in the method of the fifth implementation, the method further includes, based on the classification, extracting relevant features used in the classification, and using the extracted relevant features to update the matrix using penalization during retraining or assigning updated weights.

According to a ninth implementation of the method according to the third aspect, the method of one or more of the above implementations may further include, prior to using the trained NLP-model: selecting, as un-processed biological text, publications, portion of publications, studies, or portions of studies according to given diseases and cell types; extracting text fragments or text sections from the un-processed biological text based on an expectation that the text fragments or text sections continuing information relevant to the given diseases or cell types; and processing the extracted text fragments or text sections to generate the biomedical text, the processing comprising string matching of gene names, gene name aliases, gene products, or associated terms using a reference database.

According to a tenth implementation of the method according to the third aspect, in the method of one or more of the above implementations may further include, using the trained NLP-model may further include extracting meta information, the meta information including: publication-specification information including journal names, citations, authors or information about methods used to gather the provided information.

In an eleventh implementation of the method according to the third aspect, in the method of the tenth implementation, the associated weight may initially be determined using the meta information and one or more metrics indicating reliability including number of citations to a publication, journal quality, robustness of methods, confirmation of results in multiple publications.

According to a twelfth implementation of the method according to the third aspect, in the method of one or more of the above implementations, the grouping further includes using a language model clustering algorithm.

According to a thirteenth aspect of the present disclosure, a machine-learning system is provided, the system comprising at least one processor configured to execute the method of any one of the first through third aspects.

According to a fourteenth aspect of the present disclosure, a non-transitory computer readable storage medium is provided, which includes instructions, which when executed on one or more processors, cause the method according to any one of the first through third aspects to be executed.

Embodiments of the present disclosure address the technical problem of how to build a computer-based tool that effectively characterizes a patient's TME, diagnoses the patient, and outputs treatment protocols for cancer patents. Embodiments of the present disclosure represent important technological advancements in contrast to the state of the art, at least due to providing:
1) Increased speed and efficiency for automatically extracting relevant gene signatures from natural language, heterogeneous literature;
2) Drastically reduced workload compared to manual extraction and non-AI based automated extraction;
3) Automated screening of newly published papers;
4) Elimination of human bias compared to manual extraction;
5) Uncovering hidden or obfuscated association between cell types and gene signatures;
6) Capturing of meta information (e.g., identified by the authors), which might not be visible in the raw data;
7) An improved computer tool that characterizes a patient's TME and makes a diagnosis in a more efficient and more accurate manner than the prior art.

Therefore, embodiments of the present disclosure provide an improved special-purpose machine learning system, that is particularly configured to efficiently utilize computer resources to effectively extract pertinent information for tumor micro environment classification from vast amounts of data in the manner that no human could achieve, not only in terms of the volume and speed of processing data, but also in the ability to detect patterns and connections, free of bias. The machine-learning system further is configured to automatically analyze biological data from a patient in connection with the extracted information to not only provide a cell-by-cell characterization of the patient's tumor micro environment but to also classify the patient's disease or treatment based on such characterization. This system thus provides a machine-learning mechanism that solves the technical problems related to creating a classification tool from a vast amount of data, in view of the computing efficiency and accuracy issues inherently related therein.

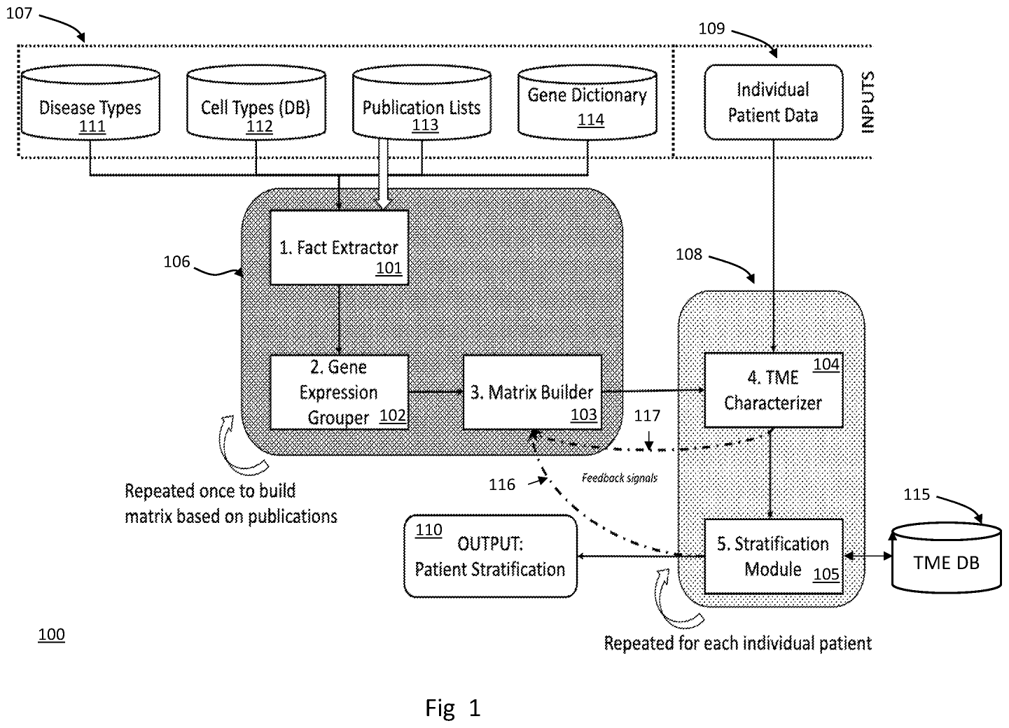

FIG. 1 illustrates a system and method implemented according to an aspect of the present disclosure.

In FIG. 1, the system and method architecture 100 includes five modules: Fact Extractor 101; Gene Expression Grouper 102; Matrix Builder 103; TME Characterizer 104; and Stratification Module 105.

The first three modules (i.e., Fact Extractor 101; Gene Expression Grouper 102; and Matrix Builder 103) are grouped as a generation set 106, which may only be executed once on a given set of input biomedical data 107 to create a disease-specific tool for classifying patients' TMEs. The input biomedical data 107 may include several databases, including databases of disease types 111, cell types 112, publication material 113, and a gene dictionary 114. As is described below, the generation set 106 uses machine-learning algorithms to efficiently extract relevant information from the input biomedical data 107, and then compile that information into a tool that provides accurate characterizations of TMEs.

The last two modules of the architecture 100 (i.e., TME Characterizer 104; and Stratification Module 105) are grouped as a characterization set 108. The characterization set 108 of modules may be run for each patient, given the individual patient data 109 input to the characterization set 108 to create the output 110 of stratifying the patient. That is, the characterization set 108 represents the execution of the machine-learning assembled tool for characterizing TMEs.

Further details of each of the modules of the architecture 100 are provided below.

The Fact Extractor 101 is a natural language processing (NLP) algorithm trained specifically on biological/biomedical data. This specific training ensures that the model can understand phrasing often used in biological texts as well as understand when specific biological or biomedical terms show up, which can consist of unusual characters or character compositions (for example chemical notations). Any language model, particularly a large language model, with the ability to classify tokens can be used to implement the Fact Extractor 101. For example, the fact extraction algorithm discussed in U.S. Pat. No. 11,741,318 may be used, or the Modular & Iterative Multilingual Open Information Extraction (MILIE) algorithm or BenchIE framework may be used, which are respectively described in Kotnis, et al., “MILIE: Modular & Iterative Multilingual Open Information Extraction,” Proceedings of the 60^{th} Annual Meeting of the Association for Computational Linguistics, 1:6939-6950, Ireland, and Gashteovski et al., “BenchIE: A Framework for Multi-Faceted Fact-Based Open Information Extraction Evaluation,” Proceedings of the 60^{th} Annual Meeting of the Association for Computational Linguistics, 1:4472-4490, Ireland. (The entirety of the listed patent and publications are hereby incorporated by reference herein).

The Fact Extractor 101 may be trained using training data, which includes sentences as inputs and facts as outputs. Each fact is a triple of the form (subject, verb, object). For example: Input sentence: “Sen. Mitchell, who is from Maine, is a lawyer.” Output triples: (“Sen. Mitchell”; “is from”; “Maine”) and (“Sen. Mitchell”; “is”; “a lawyer”).

The Fact Extractor 101 may operate on pre-selected publications/parts of publications that contain relevant information, e.g., the results sections, which may be stored in the publication material database 113. The pre-selection includes removing of reviews, e.g., types of publications that do not contain original research, and filtering for specific cancer types or disease types. Furthermore, the publication data may be pre-processed by generating tokens for extractions (e.g., groups of sentences), and by text-matching gene names by using a reference database (e.g., the gene dictionary 114), which may contain gene names, aliases, and gene product names, which can differ widely due to historical reasons. Additionally, the Fact Extractor 101 may be given a list of cell types, which also usually come from a database (e.g., cell type database 112), for example a list of known immune cells and their aliases. The Fact Extractor 101 may also use information related to individual disease types (e.g., provided in the disease type database 111), such information may provide associations between certain cell types, gene activations, and diseases.

In an exemplary embodiment, the pre-processing to generate tokens for extractions may be executed by an algorithm that extracts sentences by looking for full stops (i.e., periods) in the publications. This pre-processing algorithm could be made more sophisticated, e.g. by using regular expressions that determine whether a full stop indicates the end of a sentence or has another use (e.g. as in “Mr. Smith”). Alternatively or additionally, the pre-processing algorithm may also use chunking to split a sentence into coherent units. This could be done using regular expressions (e.g. splitting at a comma) or using more sophisticated methods, for example assign part of speech tags (verb, noun, etc.) to the words in the sentence, and using this information for chunking. See, e.g., Bachani, Chunking in NLP: decoded, Towards Data Science, April 2020, available at <<towardsdatascience.com/chunking-in-nlp-decoded-b4a71b2b4e24>> (the entire contents of which is hereby incorporated by reference herein).

Given all input information 107, the Fact Extractor 101, of the present embodiment, operates in the following manner for a given input text (e.g., a sentence or tokens). The input text is converted into a series of facts, where each fact may be saved as relational data. For example, each fact may be saved as a triple, in the form of (subject, relation, object). Additionally, meta information is collected and extracted. The meta information may include publication-specific information, such as journal, citations, authors. and information about the methods used to gather the provided information. The following is exemplary pseudo code, which may be used to implement an embodiment of the above-described operations using example data:

```
#input token list
  Input_token_example=[‘The CD4+ cells are
  characterized by a high expression of highly regulated CD4
  genes’,‘CD4 cells are more active during virus infections’]
#create a fact_extractor object from the FactExtractor class
  fact_extractor=FactExtractorClass.createObject( )
#query the fact_extractor using the input token
  output_query, additional_meta_info =
  fact_extractor.extract_information(input_token_example)
#display the first element of each of the new variables
  print(output_queries[1])
  ->[‘CD4’,‘high expression’, ‘CD4+’]
  print(additional_meta_info[1])
  ->{‘Journal’: ‘Frontiers of Immunology’, ‘Title’:
  ‘Effects of CD4+ cells on immune system’, ‘Author’: ‘John Doe’,
  ‘Year’:‘2022’, ‘Finding confidence score’: 95%]
```

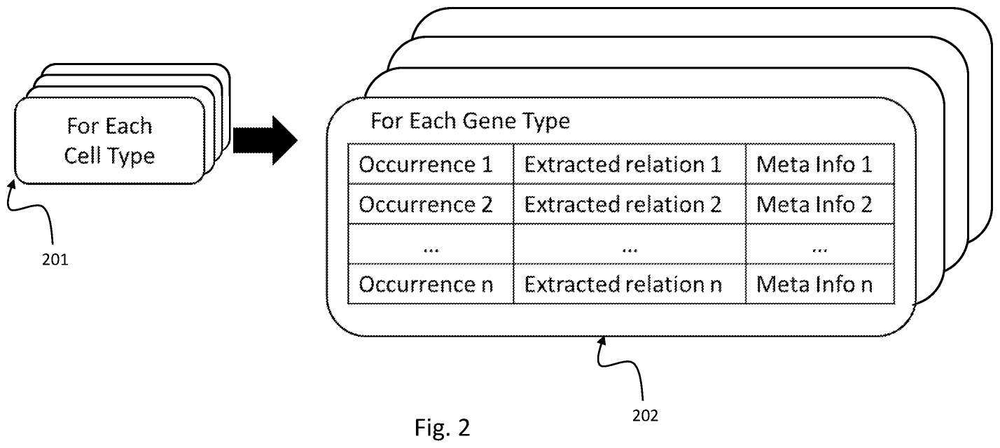

FIG. 2 illustrates an example of information extracted by the Fact Extractor 101.

As shown in FIG. 2, the Fact Extractor may create a data structure that saves the extracted facts according to each cell type processed. Thus, for each cell type 201, information is stored based on each gene type 202 found to have a relation thereto by the Fact Extractor. Ultimately, for each gene type, the extracted relational information is organized based on the occurrence, the extracted relation, and the meta information.

Returning to FIG. 1, using the information from the gene dictionary 114 (e.g., information about gene aliases and gene names), the Gene Expression Grouper 102 groups the extracted information by genes. For example, the Gene Grouper 102 may search the information provided from the Fact Extractor to find a collection of genes referenced therein (or gene product names referenced therein) that are the same, and group the extracted information received from the Fact Extractor according to such search. For each cell type, the Gene Expression Grouper 102 may also estimate an expression proxy value by taking into account if a gene is described as “expressed”, “highly expressed”, “not expressed”, etc. For example, this estimation could be made by counting all publications mentioning a target expression, and then multiplying by a factor representing “high”, “normal”, “low” or “none”, depending on the method of confirmation used. Alternatively, the Gene Expression Grouper may use a binary system, only distinguishing between “expressed” and “not expressed”, which could be implemented by counting publications mentioning a positive or negative association between gene and cell type.

The third module, the Matrix Builder 103, uses the extracted and grouped information to create a matrix with cell types as rows and genes as columns. The values are based on the expression proxy values provided by the Gene Expression Grouper. In this step, meta information is also considered, e.g., by weighting proxy values by number of occurrences, method reliability and robustness, as well as paper quality estimation metrics. For example, a low-resolution high throughput wet lab method could be assigned a lower weighting than a very specific assay confirming expression of a certain gene product. The overall value in the matrix, therefore, also reflects confidence of the gene-cell type association. It can either be calculated as one combined value or split into expression proxy and confidence by adding another dimension to the matrix. The Matrix Builder may be implemented, for example, according to the following exemplary pseudo code:

```
# Variables which are fixed for the first round
  a = 1
  b = 1
# iterate in all the cell and gene combination cited in the literature
  For (cell, gene) in zip(cells, genes):
      # extract the dataframe containing only the combination of cell
      and gene sub_df = df[“cell” = cell, “gene” = gene]
# sum of all biotechnological reliability scores which allowed to detect in
all the manuscripts the gene product related the cell
      T = sub_df[“tech_reliable_score”].sum( )
# sum of all citations number of the articles mentioning the detection of
the gene product related to the cell
      N = sub_df[“citation_nb”].sum( )
    # Calculation of the weight attributed to the gene product for the
considered cell
        W = a * N + b * T
```

The fourth module, the TME Characterizer 104, analyses the activity of the genes in each cell from a patient sample. The patient sample may be single cell RNA sequenced, creating a list of cells and their active genes describing gene activity patterns. These gene activity patterns are then matched to the matrix created by the Matrix Builder 103. Through this matching, each of the observed cells of a patient can be matched to one of the cell types that was given (or uncovered) by the Fact Extractor 101. Therefore, for each patient sample, a list of cell types and their fraction within the sequenced sample is created, which is its TME characterization. The TME Characterizer may be implemented, for example, according to the following exemplary pseudo code:

```
  #inputs are a cell-gene dictionary and the patient information
  cell_gene_dictionary=[{‘Cell’:CD8, ‘Gene’:CD4, Regulation:‘High’,
  ‘Weight’:80%},
  {‘Cell’:‘CD8’, ‘Gene’:‘CD3’, Regulation:‘High’, ‘Weight’:0.7},
  ... ,
  {‘Cell’:CD8, ‘Gene’:‘TP4’, Regulation:‘Low’, ‘Weight’:0.45}]
#data are already normalized, so we already know which genes are highly or
downregulared
  normalized_patient_12345_sc_seq_expression=[{‘Cell’:‘SC01’,‘CD4’:13.66,‘CD8’:
  0.01,‘TP4’:5.676},
  {‘Cell’:‘SC02’,‘CD4’:3.12,‘CD8’:0.1,‘TP4’:2.6},
  {‘Cell’:‘SC03’,‘CD4’:30.49,‘CD8’:9,‘TP4’:22}]
  upregulation_treshold=5
  tme_characterizer=TMECharacterizer.createObject( )
  tme_characterizer.classify_cells(cell_gene_dictionary,
  patient_12345_sc_seq_expression, upregulation_threshold, restriction=True)
#definition of the method
  def classify_cells(cell_gene_dictionary, patient_12345_sc_seq_expression,
  upregulation_threshold, restriction=True):
  classification=[ ]
  for element in patient_12345_sc_seq_expression:
    classification_dict={ }
    classification_dict[‘Cell’]=element[‘Cell’]
    for i in element.keys( ):
      if i!=‘Cell’:
        for j in cell_gene_dictionary:
          if restriction==True:
            if j[‘Gene’]== i and j[‘Weight’]>90%:
              if j[‘Regulation’]==‘High’ and
  element[i]>upregulation_threshold:
                classification_dict[i]=‘OK’
              else:
                classification_dict[‘i’]=‘Not OK’
          else
            if j[‘Gene’]== i:
              if j[‘Regulation’]==‘High’ and
  element[i]>upregulation_threshold:
                classification_dict={i}=‘OK’
              else:
                classification_dict[‘i’]=‘Not OK’
  if ‘Not OK’ not in classification_dict.elements( ):
  classification.append{‘Cell_patient’:i, ‘Type’:‘Cell’}
```

The final module, the Stratification Module 105, compares the new patient's TME characterization (e.g., as the cell type frequencies determined by the TME Characterizer) to the historical TME characterizations (which may already be stored in a database 115), which have been established by associating TME profiles to disease outcomes and treatment specifications. This historical TME information can also be provided from external resources like publications, and from samples produced within a clinical trial. This association is done by applying a supervised method to assign the patient to a specific disease group our outcome group. This association can be implemented by any type of classifier trained on known associations between cell type frequencies/ratios and tumor or disease types. The steps to train this classifier would follow standard machine learning procedures. An example for a publication that can be used to gather the target information includes that published by Bagaev et al, “Conserved pan-cancer microenvironment subtypes predict response to immunotherapy,” Cancer Cell, 39:6, 747-749, June 2021 (the entire contents of which is hereby incorporated by reference herein).

The stratification module can also extract the most relevant features used for the classification, and this information is given back to the Matrix Builder, e.g., using a feedback signal 116. This extraction may be done by estimating feature importance and returning the features that have the most influence on the patient stratification (e.g., which cell type population as part of the TME is most important to determine which group the patient belongs to).

The Matrix Builder can then be repeated by using a penalization during the retraining or reassigning weights. Additionally, the TME characterizer can also provide a feedback signal 117 to the Matrix Builder based on findings of enriched marker genes in patient sample cell types. According to an embodiment, the retraining/reassignment may be implemented according to the following exemplary pseudo code:

```
“# Retrieve the value from the TME characterizer and the stratification
module for the gene
# and cell type of interest
  tme = df[“cell” = cell, “gene” = gene][“tme_characterizer_score”]
  strat = df[“cell” = cell, “gene” = gene][“stratification_score”]
# update the value with new calculated weight for a specific cell type and
gene
  W_old = df_heatmap.loc[df_heatmap [“Cell type” = cell, “Gene
  product”=gene]][ “Weight”]
  W_new = W_old / strat[CC1] + tme
  df_heatmap.loc[df_heatmap [“Cell type” = cell, “Gene
  product”=gene]][ “Weight”] = W_new
```

Here, “strat” is that score 1 is the most important, and so the lower the rank, the less important it is, meaning that the weight needs to be lower. Also, “tme” is the intensity of the expression in the cell type from the patient, and so it needs to be added to the weight. Thus, if it is highly expresses, it has a higher weight.

In accordance with an exemplary embodiment, the three modules in the generation set 106 (i.e., Fact Extractor 101; Gene Expression Grouper 102; and Matrix Builder 103) are usually run once for a specific disease type, using a certain list of cells or cell types, a pre-defined subset of publications, a gene dictionary, or a combination of these three inputs. In contrast, for such an embodiment, the characterization set 107 (i.e., TME Characterizer 104 and Stratification Module 105) is run for each individual patient input data. The feedback signals received from the characterization set 107, however may be periodically used (e.g., for every n operations of the characterization set 107) by the Matrix Builder 103, e.g., to update the weights.

Below are provided some exemplary embodiments of a machine-learning tool implemented according to aspects of the present disclosure to extract information and to characterize tumor microenvironments of patients.

In a first embodiment, the tool may perform patient stratification based on extracted cell-gene mappings and patient information.

Use Case: Identify cancer patients who would respond to a certain treatment like a personalized cancer vaccine or might need additional treatment to stimulate their immune system.

Data Source: (1) Relevant publication of the patient's disease; (2) Patient's sequencing data.

Implementation: (1) Extract, from relevant publications, a cell-gene mapping. (2) Weigh the results to build a matrix by specific factors indicating the reliability. For example, one such factor may be that results confirmed by independent groups in journals known for thorough peer-review are valued higher than results by one group published in a lesser-known journal. Methods to detect the gene or its product used within the publication to obtain the results can also be used as a strong factor indicating reliability. Multiple methods or some specific methods lead to more accurate detection of the gene or its product and will increase the weight. (3) Patients' sequencing data is mapped against the matrix to characterize their TME to stratify how likely their response for a neoantigen vaccine would be or if additional immune stimulating drugs are required.

Output: Whether the patient should be given a cancer neoantigen vaccine.

In a second embodiment, the machine-learning tool extracts genes linked to diseases.

Use Case: Signatures of relevant genes can also be extracted for specific diseases instead of focusing on cell types. For this purpose, pre-filtering of literature according to a disease family could still be the first step, while the automated extraction and therefore association of genes could be implemented to work with more detailed disease subgroups like specific cancer subtypes instead of cell types.

Data Source: (1) Relevant publications of a particular disease or family of diseases; (2) Patient's sequencing data.

Implementation: (1) Extract expressed genes and mutations associated with a particular disease from publications. (2) Weigh results according to factors similar to the first use case above. (3) Identify the presence of mutations and overexpression of genes in patient sequencing data, e.g., DNA via whole genome sequencing (WGS), and RNA sequence

Output: Diagnosis of the patient

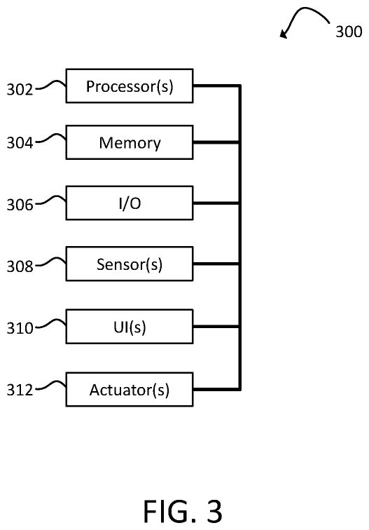

Referring to FIG. 3, a processing system 300 can include one or more processors 302, memory 304, one or more input/output devices 306, one or more sensors 308, one or more user interfaces 310, and one or more actuators 312. Processing system 300 can be representative of each computing system disclosed herein.

Processors 302 can include one or more distinct processors, each having one or more cores. Each of the distinct processors can have the same or different structure. Processors 302 can include one or more central processing units (CPUs), one or more graphics processing units (GPUs), circuitry (e.g., application specific integrated circuits (ASICs)), digital signal processors (DSPs), and the like. Processors 302 can be mounted to a common substrate or to multiple different substrates.

Processors 302 are configured to perform a certain function, method, module, or operation (e.g., are configured to provide for performance of a function, method, or operation) at least when one of the one or more of the distinct processors is capable of performing operations embodying the function, method, module or operation. Processors 302 can perform operations embodying the function, method, module or operation by, for example, executing code (e.g., interpreting scripts) stored on memory 304 and/or trafficking data through one or more ASICs. Processors 302, and thus processing system 300, can be configured to perform, automatically, any and all functions, methods, modules and operations disclosed herein. Therefore, processing system 300 can be configured to implement any of (e.g., all of) the protocols, devices, mechanisms, systems, modules, and methods described herein.

For example, when the present disclosure states that a method or module performs task “X” (or that task “X” is performed), such a statement should be understood to disclose that processing system 300 can be configured to perform task “X”. Processing system 300 is configured to perform a function, method, module, or operation at least when processors 302 are configured to do the same.

Memory 304 can include volatile memory, non-volatile memory, and any other medium capable of storing data. Each of the volatile memory, non-volatile memory, and any other type of memory can include multiple different memory devices, located at multiple distinct locations and each having a different structure. Memory 304 can include remotely hosted (e.g., cloud) storage.

Examples of memory 304 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 304.

Input-output devices 306 can include any component for trafficking data such as ports, antennas (i.e., transceivers), printed conductive paths, and the like. Input-output devices 306 can enable wired communication via USB®, DisplayPort®, HDMI®, Ethernet, and the like. Input-output devices 306 can enable electronic, optical, magnetic, and holographic, communication with suitable memory 306. Input-output devices 306 can enable wireless communication via WiFi®, Bluetooth®, cellular (e.g., LTE®, CDMA®, GSM®, WiMax®, NFC®), GPS, and the like. Input-output devices 306 can include wired and/or wireless communication pathways.

Sensors 308 can capture physical measurements of environment and report the same to processors 302. User interface 310 can include displays, physical buttons, speakers, microphones, keyboards, and the like. Actuators 312 can enable processors 302 to control mechanical forces.

Processing system 300 can be distributed. For example, some components of processing system 300 can reside in a remote hosted network service (e.g., a cloud computing environment) while other components of processing system 300 can reside in a local computing system. Processing system 300 can have a modular design where certain modules include a plurality of the features/functions shown in FIG. 3. For example, I/O modules can include volatile memory and one or more processors. As another example, individual processor modules can include read-only-memory and/or local caches.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “of” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A computer-implemented machine-learning method for characterizing a tumor micro environment, the method comprising:
using a trained natural language processing machine learning model (NLP-model), extracting facts from biomedical text, the extracted facts comprising relationship information between cell types and found gene names;
using a reference database comprising gene names and gene aliases, grouping the extracted facts according to associated genes to generate extracted and grouped information; and
generating a matrix from the extracted and grouped information with a first axis representing cell types and second axis representing genes, each value of the matrix being respectively calculated based on an importance of an associated gene taken and an associated weight, the associated weight being based on at least one of associated publication meta information or an associated detection method's robustness and reliability.

2. The method of claim 1, the method further comprising:
characterizing the tumor micro environment based on cell expression data of a patient by matching the cell expression data with the matrix.

3. The method of claim 2, the method further comprising:
receiving a biological sample of a tumor of the patient; and
using ribonucleic acid (RNA) sequencing on the biological sample, generating the cell expression data, which comprises respective active gene information associated with each cell of a plurality of cells detected in the biological sample, which in turn corresponds to expression patterns, each respective expression pattern of the expression patterns being associated with a single cell of the cells detected in the biological sample.

4. The method of claim 3, wherein characterizing the tumor micro environment based on the cell expression data of the patient by matching the cell expression data with the matrix comprises:
for each of the cells of the plurality of cells detected in the biological sample:
finding a match in the matrix for the respective expression pattern; and
assigning the respective cell to one of the cell types of the extracted facts,
generating a list of the cell types assigned to the plurality of cells detected in the biological sample;
generating cell type fraction data by determining, for each respective cell type of the cell types in the list, a fraction of the respective cell type from among the cell types;
outputting the list of the cell types and the cell type fraction data as the tumor micro environment characterization.

5. The method of claim 2, the method further comprising updating the matrix based on enriched marker genes found in the biological sample.

6. The method of claim 2, the method further comprising:
classifying, using the tumor micro environment characterization, the patient to a disease subgroup, a treatment response, adjuvant therapy recommendation, or disease outcome, treatment specification.

7. The method of claim 6, wherein the classifying comprises comparing the tumor micro environment characterization of the patient to historical tumor microenvironment characterizations.

8. The method of claim 6, wherein the classifying comprises using a trained machine-learning classification model to assign the patient to a particular classification using the tumor micro environment characterization as input.

9. The method of claim 6, the method further comprising, based on the classification, extracting relevant features used in the classification, and using the extracted relevant features to update the matrix using penalization during retraining or assigning updated weights.

10. The method of claim 1, the method further comprising, prior to using the trained NLP-model:
selecting, as un-processed biological text, publications, portion of publications, studies, or portions of studies according to given diseases and cell types;
extracting text fragments or text sections from the un-processed biological text based on an expectation that the text fragments or text sections continuing information relevant to the given diseases or cell types; and
processing the extracted text fragments or text sections to generate the biomedical text, the processing comprising string matching of gene names, gene name aliases, gene products, or associated terms using a reference database.

11. The method of claim 1, wherein using the trained NLP-model further comprises extracting meta information, the meta information comprising: publication-specification information comprising journal names, citations, authors or information about methods used to gather the provided information.

12. The method of claim 11, wherein the associated weight is initially determined using the meta information and one or more metrics indicating reliability comprising number of citations to a publication, journal quality, robustness of methods, confirmation of results in multiple publications.

13. The method of claim 1, wherein the grouping further comprises using a language model clustering algorithm.

14. A computer system comprising one or more hardware processors which, alone or in combination, are configured to provide for execution of a machine-learning method for characterizing a tumor micro environment, the method comprising:
using a trained natural language processing machine learning model (NLP-model), extracting facts from biomedical text, the extracted facts comprising relationship information between cell types and found gene names;
using a reference database comprising gene names and gene aliases, grouping the extracted facts according to associated genes to generate extracted and grouped information; and
generating a matrix from the extracted and grouped information with a first axis representing cell types and second axis representing genes, each value of the matrix being respectively calculated based on an importance of an associated gene taken and an associated weight, the associated weight being based on at least one of associated publication meta information or an associated detection method's robustness and reliability.

15. A tangible, non-transitory computer-readable medium having instructions thereon which, upon being executed by one or more hardware processors, alone or in combination, provide for execution of a machine-learning method for characterizing a tumor micro environment, the method comprising:
using a trained natural language processing machine learning model (NLP-model), extracting facts from biomedical text, the extracted facts comprising relationship information between cell types and found gene names;
using a reference database comprising gene names and gene aliases, grouping the extracted facts according to associated genes to generate extracted and grouped information; and
generating a matrix from the extracted and grouped information with a first axis representing cell types and second axis representing genes, each value of the matrix being respectively calculated based on an importance of an associated gene taken and an associated weight, the associated weight being based on at least one of associated publication meta information or an associated detection method's robustness and reliability.
